(19) United States
(12) Patent Application Publication
(10) Pub. No.: US 2007/0269418 A1
(43) Pub. Date: Nov. 22, 2007

(54) Hair Growth Inhibition

(75) Inventors: Jens Richard Pedersen, Vejle (DK); Torben Richard Pedersen, Vejle Ost (DK)

Correspondence Address: BROWDY AND NEIMARK, P.L.L.C.;624 NINTH STREET, NW, SUITE 300, WASHINGTON, DC, 20001-5303, US

(73) Assignee: Cosmedical ApS, Copenhagen K (DK)

(21) Appl. No.: 10/574,937

(22) PCT Filed: Oct. 11, 2004
(86) PCT No.: PCT/DK04/00695

§ 371 Date: Mar. 28, 2007

Related U.S. Application Data
(60) Provisional application No. 60/525,967, filed on Dec. 1, 2003.

(30) Foreign Application Priority Data
Oct. 10, 2003 (DK) PA 2003 01500

(51) Int. Cl. A61K 38/48

(52) U.S. Cl. 424/94.64; 424/94.1; 424/94.63; 424/94.65; 514/159

(57) ABSTRACT

The invention relates to the use of a composition for the preparation of a hair growth inhibitor and furthermore to the use of a system for the preparation of a hair growth inhibitor. The invention also relates to a composition comprising at least one enzyme, a solvent, and a water activity reducing agent. Moreover, the invention concerns a system for hair growth inhibition comprising a first component and a second component, wherein the first composition of the first component comprises at least one enzyme, a solvent and a water activity reducing agent, and wherein the second composition of the second component comprises an enzyme-activating agent and/or a anti-inflammatory, penetration-promoting agent and/or a preservative agent. The invention in addition relates to a method for inhibiting hair growth and a method for the preparation of a hair growth inhibitor.

### FIELD OF INVENTION

The present invention relates to a use of a composition and a system for the preparation of a hair growth inhibitor. The present invention also concerns a composition for inhibiting hair growth comprising at least one enzyme, a two-component hair growth inhibiting system comprising at least one enzyme. Furthermore, a method for inhibiting hair growth using the composition and/or the two-component system, as well as a method for producing the composition and system are described.

All patent and non-patent references cited in the application, or in the present application, are also hereby incorporated by reference in their entirety.

### BACKGROUND OF INVENTION

To meet the requirement from the public to avoid un-wanted hair on various body parts such as legs, arms, and facial region, a number of hair removing methods have been developed, such as using a shaver, wax-based removal of hairs or the like, or chemical removal agents.

The use of thiol-based depilatory agents, for removal of unwanted body and facial hair is well established in the art (see eg. U.S. Pat. No. 4,941,885; U.S. Pat. No. 4,631,064; U.S. Pat. No. 4,618,344). These agents react by reducing hair's protein disulfide bonds to sulfhydryl anions, thereby allowing easy removal of the weakened hairs when washed or wiped away. The combination of thiol compounds with high pH's results in ionized thiols and increased penetration of a reactant. Similarly, urea or other nitrogen-containing substances have been found to accelerate the action of the thio-component in hair removal.

Another approach to avoid un-wanted hair is the development of hair growth inhibitors which act on the hair follicle.

Hair grows from the hair follicle. The total number of hair follicles for an adult human is estimated at 5 million with 1 million on the head of which 100,000 alone cover the scalp. In humans, the only external regions of skin devoid of hair follicles are the palms of the hands and soles of the feet. The basic hair follicle structure remains essentially the same throughout the range of mammalian species with modifications for specialized functions.

The hair follicle can be recognized as a separate entity within the skin with formation and maintenance based on interaction between dermal and epidermal components. The hair follicle is an epithelial structure which undergoes growth cycles throughout adult life. The growth of the hair follicle down into the dermis forming a layered structure with a pigmented shaft is known as the anagen phase whereas a regression phase is known as the catagen phase in which the hair follicle shortens and a part of the hair follicle undergoes programmed cell death known as apoptosis. Also a hair follicle rest phase has been characterized and named the telogen phase (Seiberg et al. 1997. Dev. Dynamics 208, 553-564. Trypsin-induced follicular papilla apoptosis in delayed hair growth and pigmentation).

Proteolytic enzymes hydrolyze or break down proteins. Proteolytic enzymes are used in hair depilation due to their ability to degrade stem cells and hair follicles. Examples of proteolytic enzymes are papain, chymotrypsin, bromlain and papain.

Topical trypsin treatment of mice following depilation has been shown to induce apoptosis in the follicular papillae which results in delay in hair growth (Seiberg et al. 1997. WO 98/02134 describes the use of a trypsin-containing composition using as pharmaceutical vehicle non-ionic liposomes consisting of glycerol dilaurate, cholesterol-like compounds and fatty acid ethers. Mice are after wax-depilation treated with the composition and hair growth is effectively delayed and apoptosis at the follicular papillae is observed.

In EP 0622069 papain and chymotrypsin are used in a composition for permanent hair depilation. The proteolytic enzymes are stored in powder form in the presence of lactose. In a liquid solution, the enzyme activity is stabilized by the use of dithiotreitol, ethylene glycol, and ethylenediaminotetraacetic (EDTA). The solution is buffered to a suitable pH according to the proteolytic enzyme present in the solution.

Immediately before use the enzyme is dissolved in the liquid. The depilatory activity will be retained for only 7 days when the enzyme-containing solution is stored at 2-6° C. and for 4 weeks if kept at −15 to −20° C. The enzyme-containing liquid is supplied by ionophoresis.

Another approach for suppressing hair growth is presented in U.S. Pat. No. 6,375,948 in which plant extracts from the family Juniperus or malts from cereals are the major active compound. Proteolytic enzymes such as papain, chymotrypsin, bromelain, ficin, pancreatin, pepsin or trypsin may or may not be added in the composition. A number of components usually employed for cosmetics selected from the groups of oils, ethanols, thickeners, emulsifiers, humectants, colors, and perfumes etc. are also included in the composition. The hair growth inhibitor is applied to the skin of mice following shaving of a selected area of the mice. One application per day was repeated for 4 weeks.

WO 97/44005 describes a depilatory preparation that is applied to the skin by spreading. The preparation comprises proteolytic enzymes such as chymotrypsin or papain solubilized in a microemulsion. The microemulsion contains an inert organic agent and droplets of water in which the proteolytic enzymes are present. The water droplets are dispersed in the organic phase by use of lecithin as a surfactant. The organic phase is kept separate from the enzyme-containing water until use. Just prior to use the enzyme solution and the organic phase are mixed by gentle shaking to produce the active depilatory preparation. The depilatory action persists for 1-3 days if stored at 2-6° C.

### SUMMARY OF INVENTION

The present invention relates to a use of a composition and a system for the preparation of a hair growth inhibitor, wherein the degradation of the enzyme component has been reduced or even eliminated. Thus, in a first aspect the present invention relates to a use of composition comprising
at least one enzyme, said enzyme being dissolved in a solvent, and
water activity reducing agent, wherein said water activity reducing agent constitutes at least 50 percent by weight of the composition
for the preparation of a hair growth inhibitor.

By introducing a water activity reducing agent in the composition it is possible to produce a composition capable of prolonged storage without any substantial degradation of the enzyme component of the composition.

In another aspect the invention relates to a use of a system comprising
A first component comprising
   a first composition comprising
      at least one enzyme, said enzyme being dissolved in a solvent, and
      a water activity reducing agent
a second component comprising
   a second composition, comprising
      at least one enzyme-activating agent and/or
      at least one anti-inflammatory, penetration-promoting agent and/or
      a preservative agent
for the preparation of a hair growth inhibitor.

In yet another aspect the invention relates to a method for inhibiting hair growth, said method comprising the steps of
applying the composition as defined herein or the first composition of a system as defined herein to the body parts having accessible hair follicles
allowing said composition to access at least part of the accessible hair follicles thereby inhibiting hair growth.

In a further aspect the invention related to a method for the preparation of a hair growth inhibitor
providing at least one enzyme, at least one solvent, and at least one water activity reducing agent, and
mixing the at least one enzyme, the at least one solvent, and the at least one water activity reducing agent in a manner such that the water activity reducing agent constitutes at least 50 percent weight of the hair growth inhibitor.

In yet a further aspect the invention relates to a composition a composition for inhibiting hair growth comprising
at least one enzyme, said enzyme being dissolved in a solvent, and
water activity reducing agent, wherein said water activity reducing agent
constitutes at least 70 percent by weight of the composition.

In an additional aspect the invention relates to a system for inhibiting hair growth comprising
A first component comprising
   a first composition comprising
      at least one enzyme, said enzyme being dissolved in a solvent, and
      a water activity reducing agent
a second component comprising
   a second composition, comprising
      at least one enzyme-activating agent and/or
      at least one anti-inflammatory, penetration-promoting agent and/or
      a preservative agent.

### DESCRIPTION OF DRAWINGS

FIG. 1 shows a schematic presentation of a hair follicle in the main differentiated layers in a mature anagen hair follicle are shown.

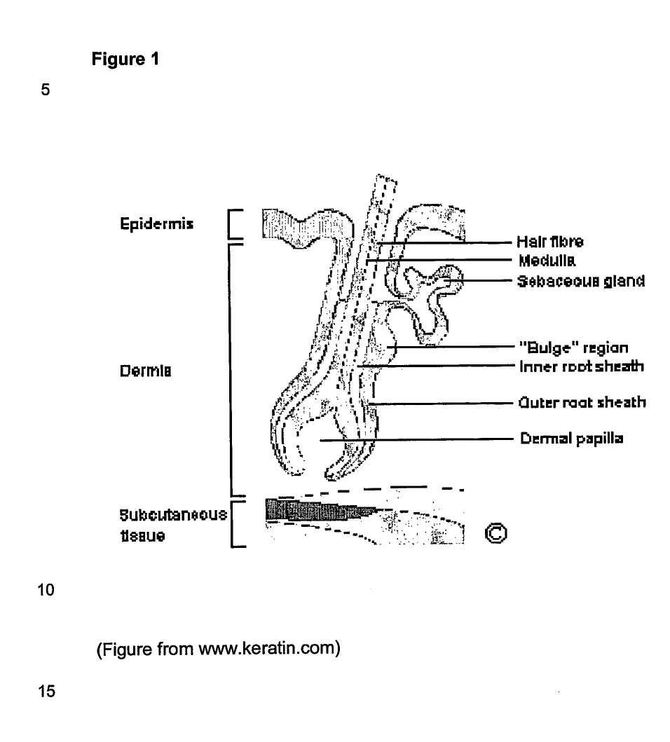

The hair follicle penetrates the dermal layer of the skin composed of fibroblast cells and collagen connective tissue interspersed with blood vessels, sweat glands and sensory nerves. The bulb region sits just above the subcutaneous (adipose fat) tissue layer. (Figure from www.keratin.com).

FIG. 2 shows an assay for proteolytic activity.

Hair inhibiting products are applied to a petridish containing casein.

Application 1: Commercial hair inhibiting product, containing papain (Surgi-Hair Stop, Ardell International). Product stored at room temperature.

Application 2: Commercial hair inhibiting product, containing papain. Product stored at room temperature (GiGi No More Hair (American International Industries).

Application 3: Composition according to this invention, comprising trypsin. Stored at room temperature for 5 months and 24 days prior to application.

FIG. 3 Photo of lower leg.

Top: Photo taken before first treatment.

Bottom: Photo taken before treatment number 4 which corresponds to 6 weeks after treatment number 3.

Treatment 1: 14 May 2003

Treatment 2: 24 Jun. 2003

Treatment 3: 5 Aug. 2003

Treatment 4: 16 Sep. 2003

FIG. 4. Photo of back.

Top: Photo taken before first treatment.

Bottom: Photo taken before treatment number 3 which corresponds to 6 weeks after treatment number 2.

Treatment 1: 2 Jul. 2003

Treatment 2: 20 Aug. 2003

Treatment 3: 1 Oct. 2003.

FIG. 5. Photo of armpit (Axilla).

Top: Photo taken before first treatment.

Bottom: Photo taken before treatment number 4 which corresponds to 9 weeks after treatment number 3.

Treatment 1: 12 Mar. 2003

Treatment 2: 7 May 2003

Treatment 3: 3 Jul. 2003

Treatment 4: 11 Sep. 2003.

FIG. 6. Photo of upper lip.

Top: Photo taken before first treatment.

Bottom: Photo taken before treatment number 6 which corresponds to 4 weeks after treatment number 5.

Treatment 1: 1 Apr. 2003

Treatment 2: 29 Apr. 2003

Treatment 3: 27 May 2003

Treatment 4: 16 Jun. 2003

Treatment 5: 14 Jul. 2003

Treatment 6: 12 Aug. 2003

### DETAILED DESCRIPTION OF THE INVENTION

### Definitions

Enzyme Activity:

Enzyme activity determined by the quantity of substrate transformed or product formed per unit time. The reaction rate that is measured depends on a number of experimental conditions such as temperature, pH, ionic strength, and the presence or absence of inhibitors or activators. It is only under the conditions specified in the prescribed assay procedure that enzyme units are defined.

The biological biopolymeric substrates (proteins, polysaccharides, bacteria, oil emulsions) often have a molecular complexity and the fact that the potential number of bonds susceptible to hydrolysis is not known and frequently varies over the time course of the assay makes a Good Standardization Practice necessary. The International Commission on Enzymes F.I.P. calculates the potency in terms of the F.I.P.—standard using a unitage expressed in “International F.I.P.—units.

The activity of a given enzyme is thus influenced by a number of parameters such as chemical and physical conditions as described above. The enzyme activity is also influenced by the action the enzyme itself, a process known as self degradation, self destruction or self digestion. Loss of enzyme activity may thus be due to any of the reasons listed.

Water Activity:

Water activity is defined as the qualitative measurement of water's energy state in a given system. The principles from thermodynamics and physical-chemical properties form the basis for this definition. The water activity of a given material depends on the types of interactions the material forms with other components in the composition such as ionic binding, dipolar forces, Van der Waals force, hydroxyl groups in sugars, carbonyl and amino groups in proteins etc. In contrast, moisture is the quantity of water in a sample measured as dry basis or moist basis which depends on the amount of material present. Consequently, water activity provides information on e.g. water migration, chemical and biochemical stability, physical properties, and shelf-life which are not provided by measurement of moisture.

Water Activity Reducing Agents:

Water activity reducing agents serve to reduce the water activity in a given composition. Water reducing agents are commonly used in foods where the agents serve to improve shelf-life of foods and to reduce the growth of pathogenic bacteria such as Clostridium botulinum in foods. Polyols such as propylene glycol, glycerol (glycerine), sorbitol, mannitol, are added to foods to provide sugar crystallization modification, water binding, or cryoprotection. Propylene glycol is primarily used as a solvent for colors, antioxidants and flavours but also serves to reduce water activity. Glycerol in foods is primarily used as plasticizer or water activity lowering compound.

Use

The present invention relates to a use of a composition or a system for the preparation of a hair growth inhibitor. The hair growth inhibitor comprises at least one enzyme, at least one solvent, and at least one water activity reducing agent as described herein. In a preferred embodiment of the invention the enzyme, solvent and water activity reducing agent is mixed.

In a further preferred embodiment the water activity reducing agent constitutes at least 50 percent by weight of the composition, such as at least 70%, such as at least 75%, such as at least 80%, such as at least 90%, such as at least 95%, such as at least 98%, such as at least 99% by weight of the composition.

Composition

The problem with prior art compositions is among others that proteolytic enzymes when stored in a solvent are degraded unless stored in a freezer. Consequently, proteolytic enzymes are often stored in powder form at temperatures below 2-6° C. or in the freezer. Furthermore, some prior art compositions need to be mixed immediately before use in order to be effective. Thus, the prior art compositions are not practical to use, in particular not practical to use by the consumer herself at home.

The present invention offers a composition that may be stored at room temperature for a prolonged period of time without degrading the enzymes, thereby facilitating the use of the composition. Accordingly, the composition according to the invention may be used both by specialists and by the normal consumer, since the product is easy to apply and requires no special storage conditions.

The advantages by the present invention have been obtained by incorporating into the composition an agent capable of reducing the water activity of the composition, whereby self-degradation of the enzyme is reduced or even absent. It is shown that when glycerol constitutes about 67 percent of the composition according to the invention the water activity is reduced to about 0.7 as measured as described herein. Furthermore, in a preferred embodiment the water activity is reduced to about 0.4 when glycerol constitutes about 83 percent by weight of the composition. Moreover, in a preferred embodiment the water activity is reduced to about 0.27 when glycerol constitutes about 92 percent by weight of the composition. In an even more preferred embodiment the water activity is about 0.25 when glycerol constitutes about 96 percent by weight of the composition. In an even more preferred embodiment the water activity is below 0.07 when glycerol constitutes 96.5 percent of the composition.

The degradation reduced by the water activity reducing agent is primarily the self-degradation known to occur in enzymes dissolved in a solvent.

The water activity reducing agent according to the invention may be any agent capable of reducing the water activity, in particular an agent capable of reducing the water activity to a value below 0.3 when measured according to the assay as described herein.

In a preferred embodiment the water activity reducing agent may be selected from the propylene glycol, glycerine, sorbitol, saccharose, and saline, or a combination thereof. In a more preferred embodiment the water activity reducing agent is glycerine.

The water activity reducing agent is present in an amount sufficient to provide the water activity reducing effect. Accordingly, it is preferred that the water activity reducing agent constitutes at least 60 percent by weight of the composition, such as at least 70 percent by weight of the composition, at least 80 percent by weight of the composition at least 85 percent by weight of the composition, at least 90 percent by weight of the composition, at least 92 percent by weight of the composition, at least 95 percent by weight of the composition.

Storage Time

The water activity reducing agent as described above is able to prolong the storage time of proteolytic enzyme, as the reduction of water activity will prevent self-destruction of the proteolytic enzyme. It is desirable for the consumer herself that the enzyme is active upon storage for a long period of time. In a preferred embodiment of the invention the storage time of the proteolytic enzyme-containing composition at room temperature is at least 12 months, such as at least 9 months, at least 6 months, at least 3 months, at least 2 months, at least 1 month, at least 3 weeks.

Proteolytic Enzyme

The composition according to the invention comprises at least one enzyme, wherein the enzyme is present in order to hydrolyze or break down proteins in the hair follicle, in particular in a hair follicle wherein the hair has been removed.

The enzyme of the composition may be any suitable enzyme, it is however preferred that the enzyme is a proteolytic enzyme.

In one embodiment of the invention the enzymeis selected from the group of serine proteases, such as trypsin, chymotrypsin, subtilisin or elastase. In another embodiment the enzyme is selected from the group of cysteine proteases, such as bromelain.

In a preferred embodiment the enzyme is selected from the group of enzymes consisting of trypsin, chymotrypsin, papain, bromelain. In a more preferred embodiment the at least one enzyme is an enzyme selected from the group of enzymes consisting of trypsin and chymotrypsin, and even more preferred the at least one enzyme is trypsin.

In a further preferred embodiment the enzyme is selected from the group of enzymes consisting of trypsin, bromelain or papain. Also preferred is the selection of at least one enzyme from bromelain or papain.

It is preferred that the enzyme constitutes at least 1.0 percent by weight of the composition, such as at least 1.5 percent by weight of the composition, such as at least 2.0 percent by weight of the composition, such as at least 2.5 percent by weight of the composition, such as at least 3.0 percent by weight of the composition. Furthermore, it is preferred that the enzyme constitutes at most 10 percent by weight of the composition. More preferably the enzyme constitutes at most 7.5 percent by weight of the composition, such as at most 6 percent by weight of the composition, such as at most 5 percent by weight of the composition, such as at most 4 percent by weight of the composition.

In a more preferred embodiment the enzyme constitutes from 2.0 to 3.0 percent by weight of the composition, such as preferably about 2.5 percent by weight of the composition, in particular when the enzyme is trypsin.

In another preferred embodiment the enzyme as described above has an activity of at least 100 F.I.P. units per gram of the composition, such as at least 150 F.I.P. units per gram of the composition, such as at least 175 F.I.P. units per gram of the composition, such as at least 200 F.I.P. units per gram of the composition, such as at least 225 F.I.P. units per gram of the composition, such as at least 250 F.I.P. units per gram of the composition.

Furthermore, it is preferred that the enzyme has an activity of at most 350 F.I.P. units per gram of the composition. More preferably the enzyme has an activity of at most 300 F.I.P. units per gram of the composition, such as 275 F.I.P. units per gram of the composition, 250 F.I.P. units per gram of the composition, such as 225 F.I.P. units per gram of the composition.

In a more preferred embodiment the enzyme has an activity from 150 to 250 F.I.P. units per gram of the composition, such as preferably about 200 F.I.P. units per gram of the composition, in particular when the enzyme is trypsin.

Solvent

The enzyme of the composition according to the invention is dissolved in a solvent. The solvent may be any suitable solvent. In a preferred embodiment the solvent is water.

In order to reduce the water activity of the composition as much as possible it is desirable to only include as little as possible of the solvent. Accordingly, it is preferred that the solvent and the enzyme constitutes at the most 20 percent by weight of the composition. More preferably the solvent and the enzyme constitute at the most 15 percent by weight of the composition, such as at the most 10 percent by weight of the composition, such as at the most 5 percent by weight of the composition such as at the most 3 percent by weight of the composition. In particular, the solvent and the enzyme constitute about 2.9 percent per weight of the composition.

Polymer

The solvent of the composition according to the invention may be cross-bound by a polymer. The polymer may be any suitable polymer. In a preferred embodiment the polymer comprises acrylic acid monomers. In an even more preferred embodiment the polymer is an acrylic acid polymer. More preferably, the polymer is selected from carbomers such carboxymethylene resins, polyacrylic acid, C10-C30 alkyl propenoate, polymer with propenoic acid, butenoic acid and/or alkyl propenoates, product with propenyl sucrose ether or propenyl 2,2-dihydroxymethyl-1.3-propanediol. In another preferred embodiment the polymer is a combination of the polymers described. In an even more preferred embodiment the polymer is is Carbopol® 940 NF polymer (Noveon, Ohio).

In order to cross-bind the solvent of the composition as effectively as desirable, it is preferred that the polymer constitutes at least 0.05 percent by weight of the composition, such as at least 0.1 percent by weight of the composition, at least 0.15 percent by weight of the composition at least 0.2 percent by weight of the composition, at least 0.25 percent by weight of the composition, at least 0.275 percent by weight of the composition, at least 0.3 percent by weight of the composition.

Furthermore, it is preferred that the polymer constitutes at most 3 percent by weight of the composition. More preferably, the polymer constitutes such as at most 2 percent by weight of the composition, such as at most 1 percent by weight of the composition, such as at most 0.5 percent by weight of the composition, such as at most 0.4 percent by weight of the composition.

In a more preferred embodiment the polymer constitutes from 0.15 to 0.25 percent by weight of the composition, such as preferably about 0.2 percent by weight of the composition, in particular when the polymer is Carbopol® 940 NF polymer (Noveon, Ohio).

Neutralization

The polymer according to the invention may be neutralized by a neutralizing agent if relevant. The neutralizing agent may be any suitable neutralizing agent, capable of neutralizing the polymer in question. In a preferred embodiment the neutralizing agent is selected from triethanolamine or diisopropanolamine. In a further preferred embodiment the neutralizing agent is diisopropanolamine.

In order to neutralize the polymer of the composition as effectively as desirable, it is preferred that the neutralizing agent constitutes at least 0.1 percent by weight of the composition, such as at least 0.2 percent by weight of the composition, at least 0.3 percent by weight of the composition, at least 0.35 percent by weight of the composition, at least 0.4 percent by weight of the composition.

Furthermore, it is preferred that the neutralizing agent constitutes at most 2 percent by weight of the composition. More preferably, the polymer constitutes such as at most 1 percent by weight of the composition, at most 0.5 percent by weight of the composition, at most 0.45 percent by weight of the composition.

In a more preferred embodiment the neutralizing agent constitutes from 0.35 to 0.45 percent by weight of the composition, such as preferably about 0.4 percent by weight of the composition, in particular when the neutralizing agent is diisopropanolamine.

Formulation

The formulation of the composition according to the invention is for topical application. Accordingly, the formulation is in a form that can be applied to the skin. In a preferred embodiment the formulation is selected from a creme, a paste, a gel, a lotion, a liquid or a liniment. In a further preferred embodiment the formulation is in the form of a creme, a gel or a paste, preferably a gel.

Two-Component System

In a preferred aspect of the invention, the ready to use two-component system according to the invention consists of two components acting in combination as a hair growth inhibitor. It is desirable that the two components are not in contact with each other prior to use in order to avoid self-destruction of the proteolytic enzymes. In a preferred embodiment the two components of the system are stored in two separate compartments. In a further preferred embodiment of the invention the two components may be placed in two separate tubes with a screw cap, two containers with a screw cap, two closed flasks or the like.

In a preferred embodiment the first component consists of the composition as described above containing at least one proteolytic enzyme, a solvent, a water activity reducing agent, and optionally a polymer, and a neutralizing agent. Preferably, the composition of the first component of the system is in the form of a creme, a gel or a paste or the like.

In a further preferred embodiment the second composition of the second component of the system according to the invention consists of at least one enzyme-activating agent and/or at least one anti-inflammatory, penetration promoting agent and/or a preservative agent.

The enzyme-activating agent of the second composition of the second component of the system activates the proteolytic enzymes of the first composition of the first component of the system. Preferably, the enzyme-activating agent is a solvent. The solvent may be any of the solvents as described above. Even more preferable, the enzyme-activating agent is water.

It is preferred that the enzyme-activating agent constitutes at least 15 percent by weight of the second composition of the system, such as at least 30 percent by weight, such as at least 40 percent by weight, such as at least 50 percent by weight, such as at least 60 percent by weight, such as at least 70 percent by weight, such as at least 80 percent by weight, such as at least 90 percent by weight, such as at least 95 percent by weight.

Furthermore, it is preferred that the enzyme-activating agent constitutes at most 98 percent of the second composition of the second component of the system, such as at most 97 percent of the second composition of the second component of the system, such as at most 96 percent of the second composition of the second component of the system, such as at most 95 percent of the second composition of the second component of the system.

In a more preferred embodiment the water-activating agent constitutes from 97 to 90 percent of the second composition of the second component of the system, such as about 97 percent of the second composition of the system, in particular when the enzyme-activating agent is water.

In order to promote the penetration of the composition into the hair follicle and to reduce potential inflammation brought about by the composition, a preferred embodiment according to the invention is to include at least one penetration-promoting, anti-inflammatory agent. In a further preferred embodiment the penetration-promoting, anti-inflammatory agent is selected from the group of salicylates, and even more preferred the penetration-promoting, anti-inflammatory agent is diethylamine salicylate.

It is preferred that the penetration-promoting, anti-inflammatory agent constitutes at least 0.5 percent by weight of the second composition of the system, such as at least 1 percent by weight, such as at least 1.5 percent by weight, such as at least 2 percent by weight of the second composition of the second component of the system.

Furthermore, it is preferred that the penetration-promoting, anti-inflammatory agent constitutes at most 5 percent of the second composition of the second component of the system, such as at most 4 percent of the second composition of the second component of the system, such as at most 3 percent of the second composition of the second component of the system.

In a more preferred embodiment the penetration-promoting, anti-inflammatory agent constitutes from 1.5 to 3 percent of the second composition of the second component of the system, such as about 2 percent of the second composition of the system, in particular when the penetration-promoting, anti-inflammatory agent is diethylamine salicylate.

In order to avoid bacterial growth and the like of the second composition of the second component of the system, a preferred embodiment is to include at least one preservative agent, and even more preferred to include sodium benzoate.

It is preferred that the preservative agent constitutes at least 0.1 percent by weight of the second composition of the system, such as at least 0.3 percent by weight, such as at least 0.5 percent by weight, such as at least 0.8 percent by weight, such as at least 0.9 percent by weight of the second composition of the second component of the system

Furthermore, it is preferred that the preservative agent constitutes at most 3 percent by weight of the second composition of the second component of the system, such as at most 2 percent by weight, such as at most 1.5 percent by weight, such as at most 1.25 percent by weight, such as at most 1.5 percent by weight of the second composition of the second component of the system.

In a more preferred embodiment the preservative agent constitutes from 0.5 to 1.5 percent of the second composition of the second component of the system, such as about 1 percent of the second composition of the system, in particular when the preservative agent is sodium benzoate.

Method for Inhibiting Hair Growth

In another aspect of the invention an individual in need of hair growth inhibition is provided a method for inhibiting hair growth. Preferably, the individual has body parts with accessible hair follicles. It is even preferred that the hair follicles are accessible due to mechanical removal of the hair from the follicle such as by wax-depilation, shaving or the like before application of the application of the hair growth inhibitor.

Furthermore, it is preferable that the enzyme-containing composition as described above is applied to the body parts having accessible hair follicles. Moreover, it is preferable that the enzyme-containing composition accesses at least some of the accessible hair follicles of the body parts.

In an even more preferred embodiment of the invention the enzyme-containing composition is allowed to access the accessible hair follicles for at least 30 seconds, such as at least 1 min, such as at least 2 min, such as at least 5 min, such as at least 10 min.

Furthermore, it is preferable that enzyme-containing composition is allowed to access the accessible hair follicles for at most 30 min, such as most 25 min, such as most 20 min, such as most 15 min.

Moreover it is preferable that the enzyme-containing composition is allowed to access the accessible hair follicles for about 30 seconds to 25 min, and in particular about 1 to 20 min when the proteolytic enzyme is trypsin.

In a preferred embodiment of the invention the enzyme-activating composition as described above is applied to the accessible hair follicles. Further, it is preferred that the enzyme-activating composition is applied subsequent to the application of enzyme-containing composition thereby inhibiting hair growth.

In an even more preferred embodiment of the invention the enzyme-activating composition is allowed to access the accessible hair follicles for at least 1 hr, such as at least 2 hrs, such as at least 3 hrs, such as at least 4 hrs, such as at least 5 hrs.

Furthermore, it is preferable that enzyme-activating composition is allowed to access the accessible hair follicles for at most 7 hrs, such as most 6 hrs.

Moreover it is preferable that the enzyme-activating composition is allowed to access the accessible hair follicles for about 4 to 7 hrs, and in particular for about 5 to 6 hrs.

It is preferable that the enzyme-containing composition and the enzyme-activating composition are applied to the skin in a thin, homogenous layer.

Furthermore, it is preferable that the enzyme--containing composition and the enzyme-activating composition are applied to the skin in amounts such as such as 3 ml per 20 cm² skin, such as 3 ml per 20 cm² skin, and in particular 1 ml per 20 cm² skin.

Moreover, the enzyme-containing composition and the enzyme-activating composition are applied to the skin in amounts such as at least 0.5 ml per 20 cm² skin, such as at least 0.75 ml per 20 cm² skin, such as at least 0.9 ml per 20 cm² skin.

Method for Producing

Another aspect of the invention relates to a method for preparation of a hair growth inhibitor comprising at least one enzyme, at least one solvent, and at least one water activity reducing agent as described above. In a preferred embodiment the enzyme, solvent and water activity reducing agent is mixed.

In a further preferred embodiment the water activity reducing agent constitutes at least 50 by weight of the hair growth inhibitor.

### EXAMPLES

### Example 1

Formulation without polymer.

Component I:

| Agent: | Percent by weight of composition |
|---|---|
| Water | 0.5 |
| Enzyme (Trypsin) | 2.4 |
| Glycerol | 97.1 |

### Example 2

Example of alternative formulation, using chymotrypsin or bromelain.

Component I:

| Agent: | Percent by weight of composition |
|---|---|
| Water | 0.5 |
| Enzyme (Chymotrypsin or | 2.4 |
| bromelain) | |
| Glycerol | 97.1 |

### Example 3

Example of two component system and formulation.

Component I:

| Agent: | Percent by weight of composition |
|---|---|
| Water | 0.5 |
| Enzyme (Trypsin) | 2.4 |
| Glycerol | 96.5 |
| Neutralizing agent | 0.4 |
| (Diisopropanolamin) | |
| Polymer (Carbomer) | 0.2 |

Component II:

| Agent: | Percent by weight of composition |
|---|---|
| Water | 97.4 |
| Polymer (Carbomer) | 0.2 |
| Neutralizing agent | 0.4 |
| (Diisopropanolamine) | |
| Penetration-promoting, | 2.0 |
| antiinflammatory agent | |
| (Diethylamine salicylate) | |

### Example 4

Alternative formulation of two-component system.

Component I:

| Agent: | Percent by weight of composition |
|---|---|
| Water | 0.5 |
| Enzyme (Trypsin) | 2.4 |
| Glycerol | 96.5 |
| Neutralizing agent | 0.4 |
| (Diisopropanolamin) | |
| Polymer (Carbomer) | 0.2 |

Component II:

| Agent: | Percent by weight of composition |
|---|---|
| Water | 98.0 |
| Penetration-promoting, | 2.0 |
| antiinflammatory Agent | |
| (Diethylamine salicylate) | |

### Example 5

Water activity (wa) measurements of the components of the system.

| Components of the system | Water % | aw |
|---|---|---|
| Component I (enzyme + glycerine) | 0.5 | 0.069 |
| Component II (enzyme activation) | 94.5 | 0.964 |
| 1:1 ratio Component I and II | 49.75 | 0.915 |
| Glycerin with water | 4.14 | 0.246 |
| Glycerin with water | 8.28 | 0.275 |
| Glycerin with water | 16.56 | 0.420 |
| Glycerin with water | 33.12 | 0.657 |

Water percentage was measured using SCALTEC SMO 01 apparatus (SCALTEC INSTRUMENTS).

Wa value measured using TESTO-400 (TESTO AG).

Water % is water in percent by weight of composition.

### Example 6

Effect on hair growth inhibition in vivo, using the composition on various body parts.

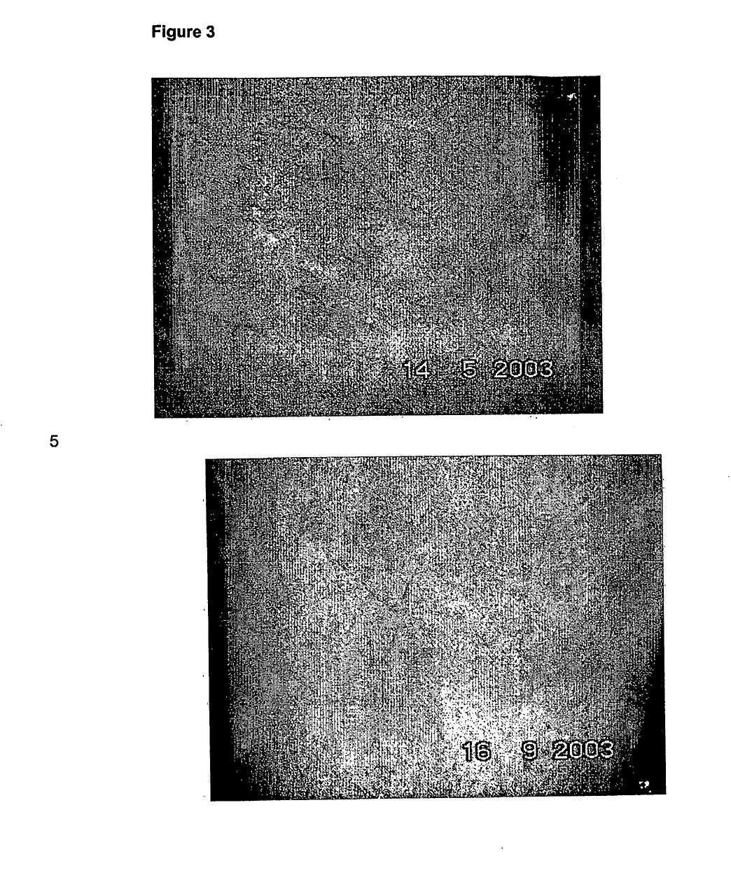

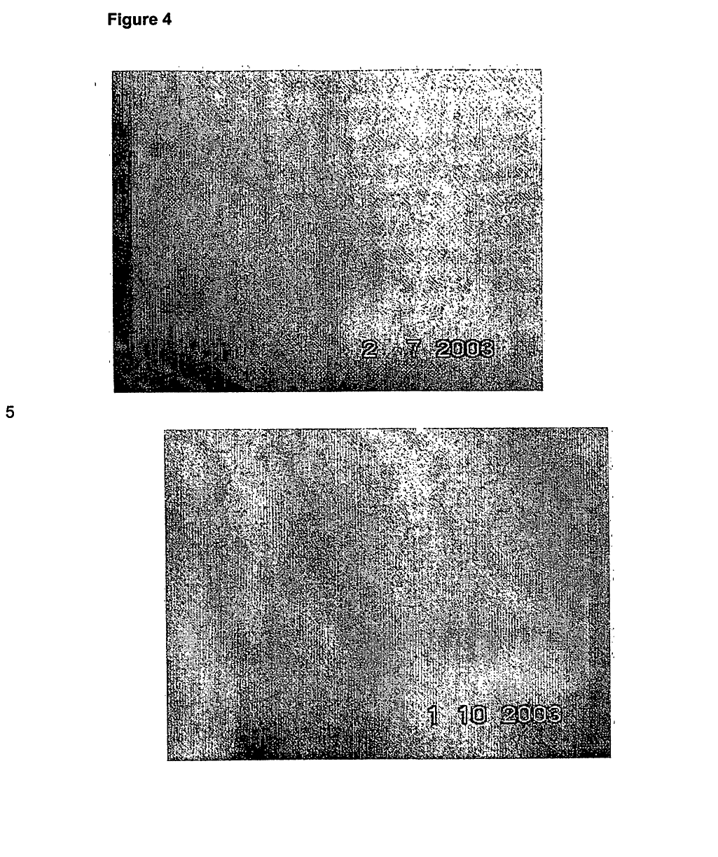

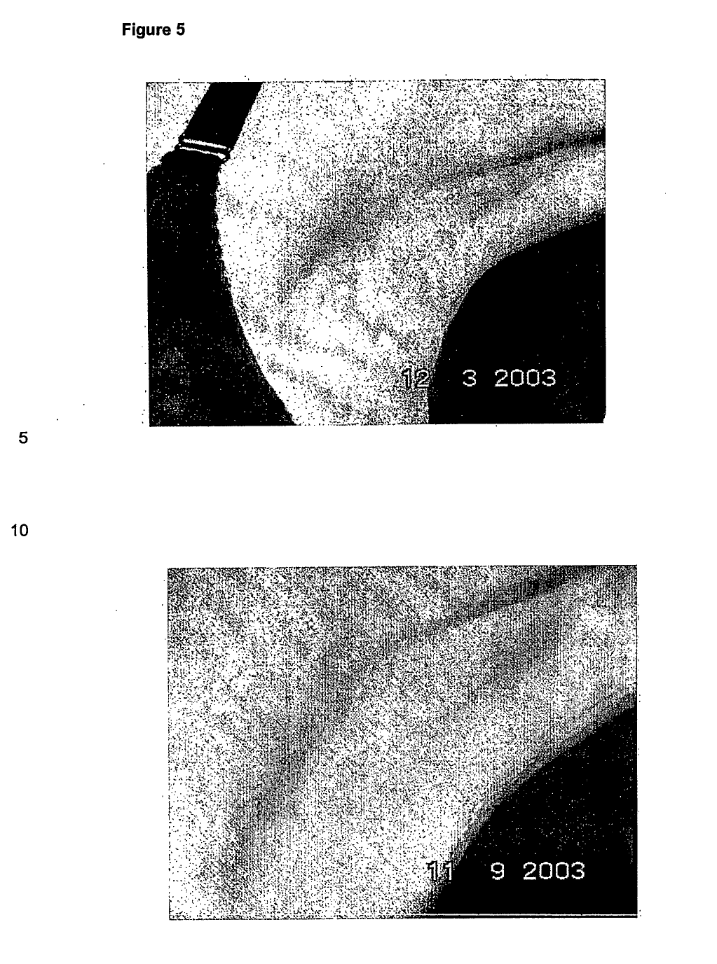

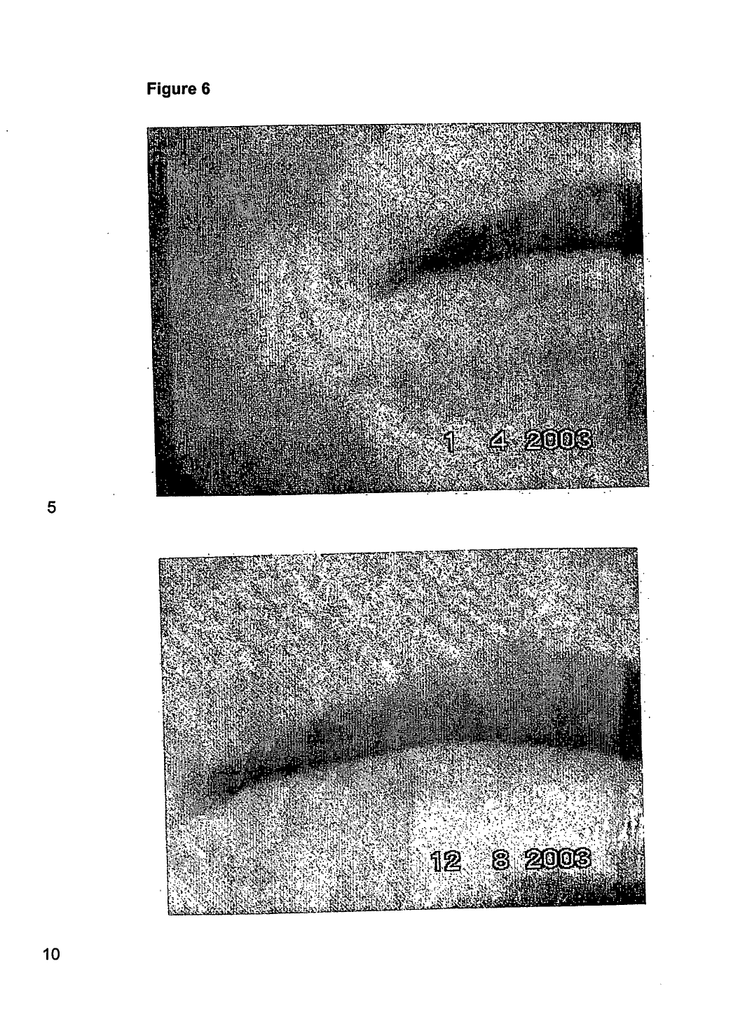

The hair growth inhibitor was applied to lower leg, back, armpit, and upper lip of humans at defined time intervals. The hair growth inhibitor was applied as a thin homogenous layer, using 1 ml per 20 cm² skin. Photographs were taken prior to the beginning of treatment and at various time points during the repeated treatments, the result of which are shown in FIGS. 3, 4, 5 and 6.

### Example 7

Measurement of activity and effect of storage in vitro:

Casein used as substrate for proteolytic degradation.

20 ml Ca-caseinate/agar solution is distributed in a petri dish (Ca-caseinate solution:

0.5 g Ca-caseinate, 1 g agar, 98.5 g 0.9% NaCl solution).10 ul of sample (diluted 1:10 in water) is loaded into 3 mm application hole. Petri dish incubated at 37° C. for 4 hrs.

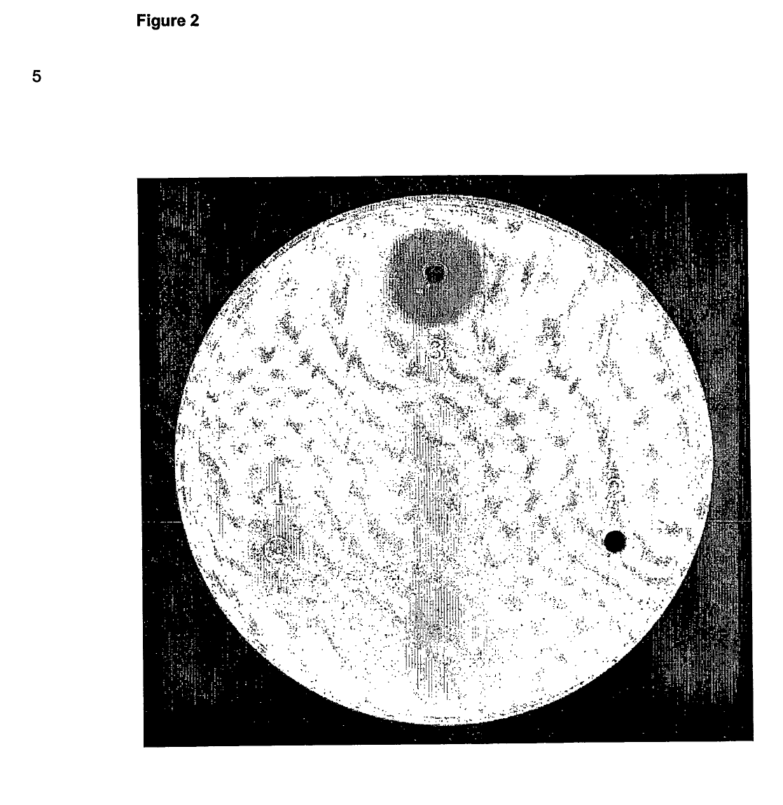

FIG. 2 shows the activity of the composition upon storage for about 6 months, using the activity measurement described above. The activity of the composition is compared to the activity of alternative commercial products.

Alternative commercial products show minimal activity, whereas the composition according to the invention displays high activity upon prolonged storage at room temperature.

Application 1: Commercial hair inhibiting product, containing papain (Surgi-Hair Stop, Ardell International). Product stored at room temperature. Application 2: Commercial hair inhibiting product, containing papain. Product stored at room temperature (GiGi No More Hair (American International Industries). Application 3: Composition according to this invention, comprising trypsin. Stored at room temperature for 5 months and 24 days prior to application.

### Example 8

The enzyme of the composition is dissolved in water. Enzyme and water constitutes 2.9 percent by weight of the composition. Water constitutes 2.4 percent by weight of the composition and enzyme constitutes 0.5 percent by weight of the composition.

1. A method for inhibiting hair growth in an individual in need thereof comprising a composition comprising at least one enzyme, said enzyme being dissolved in a solvent, and
water activity reducing agent, wherein said water activity reducing agent constitutes at least 50 percent by weight of the composition.

2. The method according to claim 1, wherein the at least one enzyme is a proteolytic enzyme.

3. The method according to claim 1, wherein the at least one enzyme is an enzyme selected from the group of enzymes consisting of trypsin, chymotrypsin, papain, and bromelain.

4-5. (canceled)

6. The method according to claim 1, wherein the solvent is water.

7. The method according to claim 1, wherein the water activity reducing agent is selected from the group consisting of glycerine, sorbitol, saccharose, and saline.

8. (canceled)

9. The method according to claim 1, wherein the water activity reducing agent constitutes at least 60 percent by weight of the composition.

10. The method according to claim 1, said composition further comprising a polymer.

11. (canceled)

12. The method according to claim 10, wherein the polymer comprises acrylic acid monomers.

13-16. (canceled)

17. The method according to claim 1, wherein the polymer constitutes 0.2 percent by weight of the composition.

18. The method according to claim 1, wherein the polymer constitutes at most 1.5 percent by weight of the composition.

19. (canceled)

20. The method according to claim 1, wherein the enzyme constitutes at least 7.5 percent by weight of the composition.

21. The method according to claim 1, said method further comprising an agent capable of neutralising the polymer.

22. The method according to claim 21, wherein the agent is diisopropanolamin.

23. The method according to claim 1, wherein the composition is in the form of a creme, a paste, a gel or a liquid.

24. (canceled)

25. A method for inhibiting hair growth in an individual in need thereof comprising a system comprising
a first component comprising
a first composition comprising
at least one enzyme, said enzyme being dissolved in a solvent, and
a water activity reducing agent
a second component comprising
a second composition, comprising
at least one enzyme-activating agent and/or
at least one anti-inflammatory, penetration-promoting agent and/or
a preservative agent
for the preparation of a hair growth inhibitor.

26. (canceled)

27. The method according to claim 25, wherein the two compositions of the two components of the system are in separate compartments.

28. The method according to claim 25, wherein the second composition of the second component of the system comprises the at least one enzyme-activating agent consisting essentially of a solvent.

29. The method according to claim 25, wherein the second composition of the second component of the system comprises the at least one enzyme-activating agent consisting essentially of water.

30. The method according to claim 25, wherein the second composition of the second component of the system further comprises at least one penetration-promoting, anti-inflammatory agent.

31. The method according to claim 25, wherein the second composition of the second component of the system further comprises the at least one penetration-promoting, anti-inflammatory agent selected from a group consisting of salicylates.

32. (canceled)

33. The method according to claim 25, wherein the second composition of the second component of the system further comprises at least one preservative agent.

34. (canceled)

35. The method according to claim 25, wherein the at least one enzyme-activating agent of the second composition of the second component of the system constitutes 95 percent by weight of the second component.

36. (canceled)

37. The method according to claim 25, wherein the at least one penetration-promoting, anti-inflammatory agent of the second composition of the second component of the system constitutes 2 percent by weight of said second composition of the second component of the system.

38. (canceled)

39. The method according to claim 25, wherein the at least one preservative agent of the second composition of the second component of the system constitutes 1 percent by weight of said second composition of the second component of the system.

40-44. (canceled)

45. A method for the preparation of a hair growth inhibitor
a. providing at least one enzyme selected from the group of enzymes consisting of trypsin, chymotrypsin, papain, and bromelain, at least one solvent, and at least one water activity reducing agent, and
b. mixing the at least one enzyme, the at least one solvent, and the at least one water activity reducing agent in a manner such that the water activity reducing agent constitutes at least 92 percent weight of the hair growth inhibitor.

46. The method according to claim 45, wherein the at least one enzyme is selected from any of trypsin, chymotrypsin, papain, and bromelain, the at least one solvent, and the at least one water activity reducing agent constitutes at least 60m percent by weight of the composition.

47. A composition for inhibiting hair growth comprising at least one enzyme selected from the group of enzymes consisting of trypsin, chymotrypsin, papain and bromelain, said enzyme being dissolved in a solvent, and
water activity reducing agent, wherein said water activity reducing agent constitutes at least 70 percent by weight of the composition.

48-51. (canceled)

52. The composition according to claim 47, wherein the solvent is water.

53. The composition according to claim 47, wherein the water activity reducing agent is selected from the group consisting of glycerine, sorbitol, saccharose, and saline.

54.-55. (canceled)

56. The composition according to claim 47, said composition further comprising a polymer.

57. (canceled)

58. The composition according to claim 56, wherein the polymer comprises acrylic acid monomers.

59.-60. (canceled)

61. The composition according to claim 47, wherein the solvent and the enzyme constitute at the most 20 percent by weight of the composition.

62. (canceled)

63. The composition according to claim 56, wherein the polymer constitutes 0.2 percent by weight of the composition.

64. The composition claim 56, wherein the polymer constitutes at most 1.5 percent by weight of the composition.

65. The composition according to claim 56, wherein the enzyme (200 F.I.P-U/g) constitutes 2.5 percent by weight of the composition.

66. The composition according to claim 56, wherein the enzyme constitutes at least 7.5 percent by weight of the composition.

67. The composition according to claim 47, said composition further comprising an agent capable of neutralising the polymer.

68. The composition according to claim 67, wherein the agent is diisopropanolamin.

69. The composition according to claim 47, wherein the composition is in the form of a creme, a paste, a gel or a liquid.

70. (canceled)

71. A system for inhibiting hair growth comprising
A first component comprising
a first composition comprising
at least one enzyme, said enzyme being dissolved in a solvent, and
a water activity reducing agent
a second component comprising
a second composition, comprising
at least one enzyme-activating agent and/or
at least one anti-inflammatory, penetration-promoting agent and/or
a preservative agent.

72. (canceled)

73. The system according to claim 71, wherein the two compositions of the two components of the system are in separate compartments.

74. The system according to claim 71, wherein the second composition of the second component of the system comprises the at least one enzyme-activating agent consisting essentially of a solvent.

75. The system according to claim 71, wherein the second composition of the second component of the system comprises the at least one enzyme-activating agent consisting essentially of water.

76. The system according to claim 71, wherein the second composition of the second component of the system further comprises at least one penetration-promoting, anti-inflammatory agent.

77. The system according to claim 71, wherein the second composition of the second component of the system further comprises the at least one penetration-promoting, anti-inflammatory agent selected from a group of salicylates.

78. (canceled)

79. The system according to claim 71, wherein the second composition of the second component of the system further comprises at least one preservative agent.

80. (canceled)

81. The system according to claim 71, wherein the at least one enzyme-activating agent of the second composition of the second component of the system constitutes 95 percent by weight of the second component.

82.-84. (canceled)

85. The system according to claim 71, wherein the at least one penetration-promoting, anti-inflammatory agent of the second composition of the second component of the system constitutes 2 percent by weight of said second composition of the second component of the system.

86. (canceled)

87. The method according to claim 1, wherein the water activity reducing agent constitutes at least 98 percent by weight of the composition.

88. The method according to claim 1, wherein the polymer constitutes at most 0.3 percent by weight of the composition.

89. The method according to claim 1, wherein the polymer constitutes at most 0.275 percent by weight of the composition.

90. The method according to claim 1, wherein the polymer constitutes at most 0.25 percent by weight of the composition.

91. The method according to claim 1, wherein the enzyme constitutes at least 6 percent by weight of the composition.

92. The method according to claim 1, wherein the enzyme constitutes at least 5 percent by weight of the composition.

93. The method according to claim 1, wherein the enzyme constitutes at least 4 percent by weight of the composition.

94. The method according to claim 1, wherein the enzyme constitutes at least 2.75 percent by weight of the composition.

95. The method according to claim 45, wherein the at least one enzyme is selected from any of trypsin, chymotrypsin, papain, and bromelain, and the at least one solvent and the at least one water activity reducing agent constitutes at least 98 percent by weight of the composition.

96. The composition according to claim 56, wherein the polymer constitutes at most 0.3 percent by weight of the composition.

97. The composition according to claim 56, wherein the polymer constitutes at most 0.275 percent by weight of the composition.

98. The composition according to claim 56, wherein the polymer constitutes at most 0.25 percent by weight of the composition.

99. The composition according to claim 56, wherein the enzyme constitutes at least 6 percent by weight of the composition.

100. The composition according to claim 56, wherein the enzyme constitutes at least 6 percent by weight of the composition.

101. The composition according to claim 56, wherein the enzyme constitutes at least 5 percent by weight of the composition.

102. The composition according to claim 56, wherein the enzyme constitutes at least 4 percent by weight of the composition.

103. The composition according to claim 56, wherein the enzyme constitutes at least 2.75 percent by weight of the composition.
